(19) United States
(12) Patent Application Publication
(10) Pub. No.: US 2014/0378929 A1
(43) Pub. Date: Dec. 25, 2014

(54) HYDROPHILIC DRESSING

(71) Applicant: Tatung Company, Taipei (TW)

(72) Inventors: Tsui-Shan Hung, Taipei (TW); Keng-Ming Chang, Taipei (TW)

(73) Assignee: Tatung Company, Taipei (TW)

(21) Appl. No.: 13/971,841

(22) Filed: Aug. 21, 2013

(30) Foreign Application Priority Data
Jun. 19, 2013 (TW) 102211449

(51) Int. Cl. A61L 15/24; A61L 15/50; A61F 13/00

(52) U.S. Cl. CPC A61L 15/24; A61F 13/00068; A61L 15/50; A61F 2013/00217
USPC 604/372

(57) ABSTRACT

A hydrophilic dressing including a base layer, a hydrophilic dressing layer and an anti-sticking layer is provided. The hydrophilic dressing layer is disposed on the base layer. The anti-sticking layer covers on a part of the hydrophilic dressing layer. While the hydrophilic dressing layer absorbs a great amount of exudate from wound, the anti-sticking layer of the hydrophilic dressing can separate avalanched hydrophilic dressing from skin. Therefore, when the hydrophilic dressing is removed, the avalanched hydrophilic dressing would not remain on the skin.

### CROSS-REFERENCE TO RELATED APPLICATION

This application claims the priority benefit of Taiwan application serial no. 102211449, filed on Jun. 19, 2013. The entirety of the above-mentioned patent application is hereby incorporated by reference herein and made a part of this specification.

### BACKGROUND OF THE INVENTION

1. Field of the Invention

The invention relates to a hydrophilic dressing, and more particularly, to a hydrophilic dressing with anti-sticking capability.

2. Description of Related Art

Generally, a dressing is mainly composed by natural plant fibers such as gauze, cotton, cotton pad and so on. When in use for a wound, the dressing can be easily stuck on the wound, such that a secondary damage to skin may occur when a newly grown skin tissue is torn off when removing the dressing. With improvement in technology and life quality, various hydrophilic dressings have been circulated in the market, including dressings such as carboxymethyl cellulose with high water absorption, hydrogel, foam and calcium alginate, so that a better wound care may be provided.

The hydrophilic dressings (with materials such as carboxymethyl cellulose, hydrogel, foam and calcium alginate) includes characteristics of higher absorption to a wound exudate while maintaining moist of the wound, such that a moist environment can be provided for the wound to heal. Further, the hydrophilic dressings with stickness may adhere evenly to the wound, thus the materials as mentioned above are major materials for the hydrophilic dressing. However, when in use for the wound with high exudate, the hydrophilic dressings are easily remained on the skin since the hydrophilic dressing may easily expand and avalanche after absorbing fluid from the wound.

Moreover, when in use for the wound without any exudate, the hydrophilic dressings may overly absorb the wound exudate due to its high absorption, so that a replacement of the hydrophilic dressings is constantly required.

In addition, a hydrophilic dressing is usually fabricated in a roll shape, so that the hydrophilic dressing may be stacked with a previous layer, such that the hydrophilic dressing may be easily stuck on a previous base layer. In subsequent processing for the hydrophilic dressing, different layers of the hydrophilic dressing need to be separated first, so it is difficult for a direct process to be performed.

### SUMMARY OF THE INVENTION

The invention is directed to a hydrophilic dressing capable of lowering a possibility of avalanched hydrophilic dressing remained on the skin, preventing wound exudate from being overly absorbed, and increasing processing efficiency by fabricating the hydrophilic dressing in a roll shape to lower a possibility of the hydrophilic dressing being stuck on a previous base layer.

A hydrophilic dressing including a base layer, a hydrophilic dressing layer and an anti-sticking layer is provided. The hydrophilic dressing layer is disposed on the base layer. The anti-sticking layer covers on a part of the hydrophilic dressing layer.

In an embodiment of the invention, a material of the anti-sticking layer is a polymer or a polymer and metal complex film.

In an embodiment of the invention, the polymer or the polymer and metal complex film includes polyethylene, polypropylene, linear low density polyethylene, nylon or high-density polyethylene, or above-said materials containing silver.

In an embodiment of the invention, the anti-sticking layer is distributed in meshes or holes on the hydrophilic dressing layer.

In an embodiment of the invention, a thickness of the anti-sticking layer 120 is approximately 0.1 to 2 millimeters.

In an embodiment of the invention, a weight per unit area of the anti-sticking layer is approximately 10 to 25 G/m².

In an embodiment of the invention, the anti-sticking layer includes a plurality of discontinuous patterns in stripes or dots.

In an embodiment of the invention, a material of the anti-sticking layer includes a compound of a polymer and silver, so that an antibacterial effect may be achieved.

In an embodiment of the invention, the hydrophilic dressing further comprises a pressure sensitive adhesive layer, in which the hydrophilic dressing layer partially covers on the base layer, and the pressure sensitive adhesive layer exposes to an area where the hydrophilic dressing layer is not covered on the base layer.

In an embodiment of the invention, the hydrophilic dressing further comprises a release layer covering on the pressure sensitive adhesive layer, the hydrophilic dressing layer and the anti-sticking layer.

In an embodiment of the invention, an outer profile of the anti-sticking layer is identical to an outer profile of the hydrophilic dressing layer.

In summary, the hydrophilic dressing of the invention provides the anti-sticking layer covering on the part of the hydrophilic dressing layer, in which the anti-sticking layer may be in meshes, holes, or includes a plurality of discontinuous patterns in stripes or dots. When the hydrophilic dressing is attached to the wound, the hydrophilic dressing layer and the wound are spaced apart by the anti-sticking layer, the wound exudate is only absorbed by the hydrophilic dressing layer through the holes of the anti-sticking layer after reaching a specific amount, so as to prevent the hydrophilic dressing layer from overly absorb the wound exudate, such that dressing replacement may be saved. Further, while the hydrophilic dressing layer absorbs a great amount of exudate from wound, the anti-sticking layer can separate avalanched hydrophilic dressing from skin so as to prevent the avalanched hydrophilic dressing from remaining on the skin. In addition, when fabricating the hydrophilic dressing, the hydrophilic dressing is rolled into a roll shape, such that the anti-sticking layer may prevent the hydrophilic dressing layer from sticking with the upper base layer.

In order to make the aforementioned and other features and advantages of the present invention more comprehensible, several embodiments accompanied with figures are described in detail below.

### BRIEF DESCRIPTION OF THE DRAWINGS

FIG. 1A is a schematic cross-sectional view of a hydrophilic dressing according to an embodiment of the invention.

FIG. 1B is a schematic view from another perspective of the hydrophilic dressing depicted in FIG. 1A after a release layer is torn off.

FIG. 2 is a schematic view of a hydrophilic dressing according to another embodiment of the invention.

FIG. 3 is a schematic view of a hydrophilic dressing according to another embodiment of the invention.

FIG. 4 is a fabricating process of the hydrophilic dressing depicted in FIG. 1.

### DESCRIPTION OF THE EMBODIMENTS

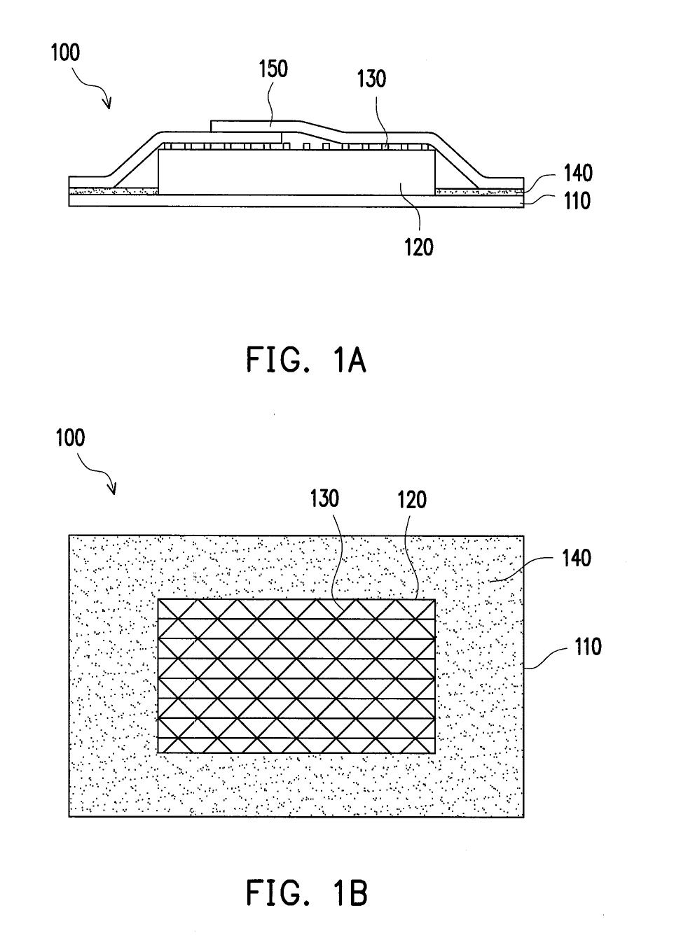

FIG. 1A is a schematic cross-sectional view of a hydrophilic dressing according to an embodiment of the invention. FIG. 1B is a schematic view from another perspective of the hydrophilic dressing depicted in FIG. 1A after a release layer is torn off. Referring to FIG. 1A and FIG. 1B, a hydrophilic dressing 100 of the present embodiment includes a base layer 110, a hydrophilic dressing layer 120, an anti-sticking layer 130, a pressure sensitive adhesive 140 and a release layer 150.

The base layer 110 is mainly configured to provide waterproof characteristic, and a material of the base layer 110 may be polyethylene, polypropylene, polyester, polycarbonate, polyurethanes, derivatives thereof or a combination thereof, but the material of the base layer 110 is not limited thereto.

The hydrophilic dressing layer 120 is disposed on the base layer 110. In the present embodiment, the hydrophilic dressing layer 120 covers a center area of the base layer 110. Certainly, in other embodiments, the hydrophilic dressing layer 120 may also cover the base layer 110 entirely. The hydrophilic dressing layer 120 is composed by material with characteristic of exudate absorption, such as hydrogel, acrylic gel, polyurethane gel (PU gel), polyurethane foam, carboxymethyl cellulose or calcium alginate, but types of the hydrophilic dressing layer 120 are limited thereto.

The hydrophilic dressing layer 120 has characteristics of providing a moist environment for a wound to heal, and absorbing exudate. The base layer 110 has characteristics of vapor transmission and waterproof. When the hydrophilic dressing 100 covers on a surface of the wound, the vapor generated in the wound may be released to the outside, and at the same time, the wound is protected from getting wet.

Therefore, the wound may remain dry during healing to reduce the probability of an infection. In the present embodiment, a thickness of the hydrophilic dressing layer 120 is approximately 0.1 to 2 millimeters.

Moreover, in the present embodiment, the anti-sticking layer 130 is covered on a part of the hydrophilic dressing layer 120, and a weight per unit area of the anti-sticking layer 130 is approximately 10 to 25 g/m². In the present embodiment, a material of the anti-sticking layer 130 is a polymer film or a polymer and metal complex film, including polyethylene, polypropylene, linear low density polyethylene, nylon or high-density polyethylene, or above materials containing silver, but the material of the anti-sticking layer 130 is not limited thereto. The anti-sticking layer 130 of the present embodiment is flexible and bendable to be attached firmly with the anti-sticking layer 120. In the present embodiment, an outer profile of the anti-sticking layer 130 is identical to an outer profile of the hydrophilic dressing layer 120.

The pressure sensitive adhesive 140 exposes to an area where the hydrophilic dressing layer 120 is not covered on the base layer 110, that is, an edge area of the base layer 110. The pressure sensitive adhesive 140 is configured to adhere the hydrophilic dressing 100 to the skin. The pressure sensitive adhesive 140 may be disposed on the area where the hydrophilic dressing layer 120 is not covered on the base layer 110. Alternatively, the pressure sensitive adhesive 140 may also be disposed on the base layer 110 entirely, and the hydrophilic dressing layer 120 may be partially disposed on the pressure sensitive adhesive 140. In other words, with such disposition, not only pressure sensitive adhesive 140 located at the edge area may adhere the hydrophilic dressing 100 to the skin, but the hydrophilic dressing layer 120 may also increase an adhesion force to the base layer 110 through the pressure sensitive adhesive 140 at the center area.

The release layer 150 covers on the hydrophilic dressing layer 120, the anti-sticking layer 130 and the pressure sensitive adhesive 140, and the release layer 150 may be torn off from the hydrophilic dressing layer 120, the anti-sticking layer 130 and the pressure sensitive adhesive 140. More specifically, when the hydrophilic dressing layer 120 is not in use, the release layer 150 covers the hydrophilic dressing layer 110, the anti-sticking layer 130 and the pressure sensitive adhesive 140, so as to prevent the hydrophilic dressing layer 120 and the pressure sensitive adhesive 140 from outside contamination or losing stickiness. When the hydrophilic dressing 100 is in use, the pressure sensitive adhesive 140 located at the edge area may fix the hydrophilic dressing 100 on the skin around the wound by tearing off the release layer 150 and aiming the wound to the hydrophilic dressing layer 120 and the anti-sticking layer 140 at the center area.

As shown in FIG. 1B, in the present embodiment, the hydrophilic dressing 100 is roughly rectangular, but a shape of the hydrophilic dressing 100 is not limited thereto. That is, in other embodiments, the hydrophilic dressing 100 may also be roughly a square or other appropriate shapes.

In the present embodiment, the anti-sticking layer 130 is disposed on the hydrophilic dressing layer 120, so that the skin and the wound contact the anti-sticking layer 130. Since a wound exudate is only absorbed by the hydrophilic dressing layer 120 though a hole portion of the anti-sticking layer 130 when the wound exudate reaches a specific amount, the hydrophilic dressing 100 is not required to be constantly replaced.

In addition, the hydrophilic dressing 100 of the present embodiment is placed on the part of the hydrophilic dressing layer 120 through the anti-sticking layer 130, so that the wound exudate may enter the hydrophilic dressing layer 120 through the hole portion of the anti-sticking layer 130. Moreover, since the hydrophilic dressing layer 120 (for absorbing the wound exudate) and the wound are spaced apart by the anti-sticking layer 130, a possibility of avalanched hydrophilic dressing being stuck on the wound can be lowered when removing the hydrophilic dressing.

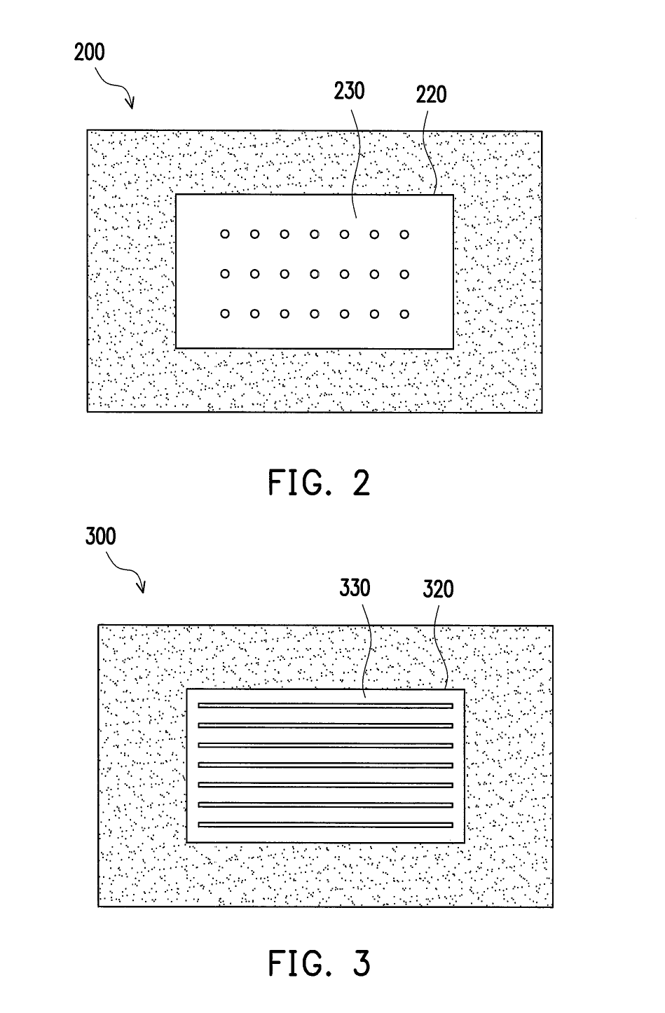

FIG. 2 is a schematic view of a hydrophilic dressing according to another embodiment of the invention. FIG. 3 is a schematic view of a hydrophilic dressing according to another embodiment of the invention. Referring to FIG. 2 and FIG. 3, a major difference between hydrophilic dressing 200 and 300 depicted in FIG. 2 and FIG. 3 and the hydrophilic dressing 100 depicted in FIG. 1 is that, the anti-sticking layer 130 is distributed on the hydrophilic dressing layer 120 in meshes in FIG. 1. In FIG. 2 and FIG. 3, anti-sticking layers 230 and 330 respectively include a plurality of discontinuous patterns in stripes or dots, so that the wound exudate may enter the hydrophilic dressing layers 220 and 320 from a dot portion or a stripe portion of the anti-sticking layers 230 and 330, respectively. Certainly, the patterns of the anti-sticking layers 230 and 330 on the hydrophilic dressing layers 220 and 320 are not limited thereto.

Patterns used to distribute the anti-sticking layers 230 and 330 on the hydrophilic dressing layers 220 and 320 are not particularly limited, as long as the anti-sticking layers 230 and 330 may cover on the hydrophilic dressing layers 220 and 320, so the wound exudate may enter the hydrophilic dressing layers 220 and 320 from hole portions of the anti-sticking layers 230 and 330, and a possibility of avalanched hydrophilic dressing being stuck on the wound can be lowered when removing the hydrophilic dressing 200 and 300.

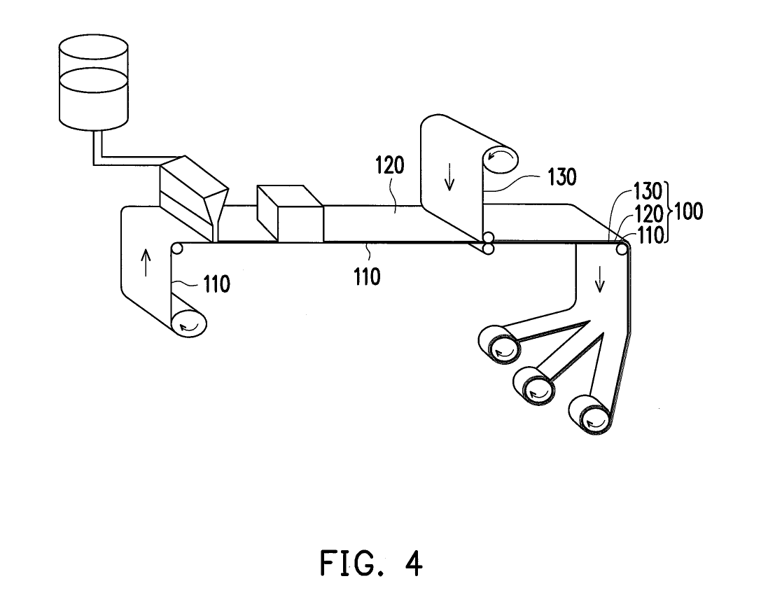

FIG. 4 is a fabricating process of the hydrophilic dressing according to an embodiment of the invention. FIG. 4 illustrates an example of fabricating the hydrophilic dressing depicted in FIG. 1, but the invention is not limited thereto. Referring to FIG. 4, first, a base layer 110 passes a working area after being released from an unwinding mechanism. Subsequently, in the working area, a hydrophilic dressing is coated on the base layer 110 though a coating machine. Next, a hydrophilic dressing layer 120 is formed by drying and ripening the hydrophilic dressing on the base layer 110. Subsequently, an anti-sticking layer 130 adheres on the hydrophilic dressing layer 120 after being released from the unwinding mechanism, so as to form a hydrophilic dressing 100. Next, processes to the hydrophilic dressing 100 are performed. In the present embodiment, processes to the hydrophilic dressing 100 refers to slitting, winding and cutting the hydrophilic dressing 100, but types of the processes to the hydrophilic dressing 100 are not limited thereto.

In the hydrophilic dressing 100 of the present embodiment, the anti-sticking layer 130 partially covers on the hydrophilic dressing layer 120, thus when winding, since the hydrophilic dressing layer 120 is covered with the anti-sticking layer 130, the hydrophilic dressing layer 120 may be protected from sticking with the upper base layer 110. Therefore, an additional step of separating different layers of the hydrophilic dressing 100 is not required, such that subsequent processing time may then be shorten.

In summary, the hydrophilic dressing of the invention provides the anti-sticking layer covering on the hydrophilic dressing layer, in which the anti-sticking layer may be in meshes, holes, or includes a plurality of discontinuous patterns in stripes or dots. When the hydrophilic dressing is attached to the wound, the hydrophilic dressing layer and the wound are spaced apart by the anti-sticking layer, the wound exudate is only absorbed by the hydrophilic dressing layer through the holes of the anti-sticking layer after reaching a specific amount, so as to prevent the hydrophilic dressing layer from overly absorb the wound exudate, such that dressing replacement may be saved. Further, while the hydrophilic dressing layer absorbs a great amount of exudate from wound, the anti-sticking layer can separate avalanched hydrophilic dressing from skin so as to prevent the avalanched hydrophilic dressing from remaining on the skin. In addition, when fabricating the hydrophilic dressing, the hydrophilic dressing is rolled into a roll shape, such that the anti-sticking layer may prevent the hydrophilic dressing layer from sticking with the upper base layer.

Although the invention has been described with reference to the embodiments thereof, it will be apparent to one of the ordinary skills in the art that modifications to the described embodiments may be made without departing from the spirit of the invention. Accordingly, the scope of the invention will be defined by the attached claims not by the above detailed description.

What is claimed is:

1. A hydrophilic dressing, comprising:
a base layer;
a hydrophilic dressing layer disposed on the base layer; and
an anti-sticking layer covering on a part of the hydrophilic dressing layer.

2. The hydrophilic dressing of claim 1, wherein a material of the anti-sticking layer is a polymer or a polymer and metal complex film.

3. The hydrophilic dressing of claim 1, wherein the polymer or the polymer and metal complex film includes polyethylene, polypropylene, linear low density polyethylene, nylon or high-density polyethylene, or above-said materials containing silver.

4. The hydrophilic dressing of claim 1, wherein the anti-sticking layer is distributed in meshes or holes on the hydrophilic dressing layer.

5. The hydrophilic dressing of claim 1, wherein an outer profile of the anti-sticking layer is identical to an outer profile of the hydrophilic dressing layer.

6. The hydrophilic dressing of claim 1, wherein a thickness of the hydrophilic dressing layer is approximately 0.1 to 2 millimeters.

7. The hydrophilic dressing of claim 1, wherein a weight per unit area of the anti-sticking layer is approximately 10 to 25 g/m².

8. The hydrophilic dressing of claim 1, wherein the anti-sticking layer comprises a plurality of discontinuous patterns in stripes or dots.

9. The hydrophilic dressing of claim 1, further comprising a pressure sensitive adhesive layer, wherein the hydrophilic dressing layer partially covers on the base layer, and the pressure sensitive adhesive layer exposes to an area where the hydrophilic dressing layer is not covered on the base layer.

10. The hydrophilic dressing of claim 9, further comprising a release layer covering on the pressure sensitive adhesive layer, the hydrophilic dressing layer and the anti-sticking layer.
